(19) United States
(12) Patent Application Publication
(10) Pub. No.: US 2007/0036859 A1
(43) Pub. Date: Feb. 15, 2007

(54) Sustained release antihistamine and decongestant composition

(76) Inventors: Ronald L. Perry, Paw Paw, MI (US); Jack T. Irwin, Kalamazoo, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/502,114

(22) Filed: Aug. 10, 2006

Related U.S. Application Data
(60) Provisional application No. 60/707,267, filed on Aug. 11, 2005.

(51) Int. Cl. A61K 31/137; A61K 9/24

(52) U.S. Cl. 424/471; 514/649

(57) ABSTRACT

A controlled-release, non-sedating antihistamine and decongestant composition which provides a 24-hour decongestant dissolution profile using standard ungranulated xanthan gum as the sole controlled-release agent and a process for preparing the same is provided. The pharmaceutical composition of the present invention typically includes: a compressed extended-release core comprising a pharmaceutically effective amount of decongestant, ungranulated xanthan gum, one or more binders, a flow agent, and a lubricant. An immediate-release coating composition is disposed on the core that typically includes a non-sedating antihistamine and at least one coating agent.

### CROSS-REFERENCE TO RELATED APPLICATION

This application claims priority under 35 U.S.C. § 119(e) and the benefit of U.S. Provisional Application No. 60/707,267 entitled SUSTAINED RELEASE ANTIHISTAMINE AND DECONGESTANT COMPOSITION, filed on Aug. 11, 2005, by Ronald L. Perry and Jack T. Irwin, the entire disclosure of which is incorporated herein by reference.

### BACKGROUND OF THE INVENTION

In order for a once-a-day formulation containing a non-sedating antihistamine, such as loratadine, and a decongestant, such as pseudoephedrine, to be effective, it must provide a decongestant dissolution profile for periods longer than 12 hours. The safety and effectiveness of the antihistamine and decongestant should also not be affected.

Loratadine is a non-sedating, long-acting tricyclic antihistamine which has been typically administered for alleviating seasonal allergic rhinitis symptoms, such as sneezing and itching. Loratadine is available in the form of conventional tablets which release loratadine by disintegration and dissolution. Typically, loratadine begins to illicit its antihistaminic effect within one to three hours after ingestion and the effect lasts in excess of 24 hours. Accordingly, loratadine 10 mg tablets are typically orally administered only once a day.

Pseudoephedrine and its pharmaceutically acceptable salts are well recognized by those skilled in the art as safe and effective nasal and ocular decongestants. Pseudoephedrine is available in the form of conventional tablets which release pseudoephedrine by disintegration and dissolution. Typically, pseudoephedrine tablets are administered orally three or four times a day for the relief of nasal congestion. However, controlled-release tablets which release a decongestant, such as pseudoephedrine, at a controlled rate such that the tablets are administered twice daily are also available.

Xanthan gum is a high molecular weight polysaccharide. Xanthan gum is generally considered to be non-gelling and must be combined with a galactomannan or a glucomannan to form a gel. Xanthan gum may also contain cellulase, which prevents its use with cellulose derivatives. Pharmaceutical mixtures using standard ungranulated xanthan gum exhibit poor tabletability. Accordingly, prior art compositions which use xanthan gum generally use either pregranulated xanthan gum or granulate the xanthan gum after adding it to a mixture including a decongestant.

There is a significant need for a once daily controlled-release non-sedating antihistamine and decongestant composition which is easily manufactured.

### SUMMARY OF THE INVENTION

One embodiment of the present invention includes a controlled-release, non-sedating antihistamine and decongestant composition which provides a 24-hour decongestant dissolution profile using standard ungranulated xanthan gum as the sole controlled-release agent for the decongestant and a process for preparing the same. The pharmaceutical composition of the present invention typically includes: a compressed extended-release core comprising a pharmaceutically effective amount of decongestant, ungranulated xanthan gum, one or more binders, a flow agent, and a lubricant. An immediate-release coating composition is disposed on the core that typically includes a non-sedating antihistamine and at least one coating agent.

Applicants have also discovered a process for preparing an extended-release decongestant, such as pseudoephedrine sulfate, and antihistamine, such as loratadine, tablet. The process of the present invention generally includes granulating a decongestant and one or more binders together to form a decongestant granulation; combining the decongestant granules with a flow agent, one or more binders, a lubricant, and ungranulated xanthan gum to form a core mixture; compressing the core mixture to form an extended-release core; thereafter coating the extended-release core with an immediate-release coating composition comprising a non-sedating antihistamine and at least one coating agent; and optionally applying a final finish coating.

### DETAILED DESCRIPTION OF THE PREFERRED EMBODIMENT

In a preferred embodiment of the present invention, the pharmaceutical composition includes a compressed extended-release core comprising: a decongestant granulation that typically includes a binder, such as microcrystalline cellulose, a decongestant (typically, a pharmaceutically acceptable pseudoephedrine salt, such as pseudoephedrine sulfate, and/or phenylephrine hydrochloride) or mixtures thereof; ungranulated xanthan gum, one or more binders, a flow agent, and a lubricant. The core is typically then coated with an immediate-release antihistamine coating comprising an antihistamine, such as loratadine or desloratadine, and at least one coating agent. Other antihistamines that may be utilized include H1 antagonist antihistamines including: ethylenediamines, such as mepyramine (pyrilamine) and antazoline; ethanolamines, such as diphenhydramine, carbinoxamine, doxylamine, clemastine, dimenhydrinate; alkylamines, such as pheniramine, chlorphenamine (chlorpheniramine), dexchlorphenamine, brompheniramine, triprolidine; piperazines, such as hydroxyzine and meclizine; tricyclics, such as promethazine, alimemazine (trimeprazine), cyproheptadine, azatadine; acrivastine; astemizole; cetirizine, levocetirizine, fexofenadine, loratadine, desloratadine, mizolastine, and terfenadine.

Decongestants are medicines used to relieve nasal congestion caused by swelling of the membranes lining the nose. Decongestants relieve the swelling by reducing the blood supply to the swollen membranes, causing the membranes to shrink. Although any suitable decongestant can be used, the preferred decongestants of the present invention are pseudoephedrine, a pharmaceutically acceptable pseudoephedrine salt, and mixtures thereof, as well as a phenylephrine salt. Pseudoephedrine is a sympathomimetic amine. Any suitable pseudoephedrine salt may be used in the present invention, however pseudoephedrine hydrochloride, (+)−pseudoephedrine sulfate, and/or phenylephrine salt such as phenylephrine hydrochloride, are typically used. Other suitable pseudoephedrine salts include sodium, hydrofluoric, sulfuric, sulfonic, tartic, fumaric, hydrobromic, glycolic, citric, maleic, phosphoric, succinic, acetic, nitric, benzoic, ascorbic, p-toluene, benzenesulfonic, naphthalenesulfonic, propionic, and the like. In addition to pseudoephedrine, other suitable decongestants include oxymetazoline, phenylpropanolamine, and other sympathomimetic drugs. Decongestants that may be utilized include, but are not limited to, those sympathominetic aminies with the following structure: 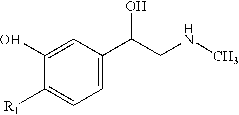
where R₁ is H or OH

Typically, the decongestant is present in the pharmaceutical composition in an amount from about 20% to about 30% by weight of the pharmaceutical composition, more typically from about 20% to about 25%, and most typically from about 22% to about 24% decongestant.

In addition to a decongestant, the decongestant granulation of the present invention also includes a substantially dry binder. Typically, the substantially dry binder is a microcrystalline cellulose, such as AVICEL®, a microcrystalline cellulose sold by FMC Corporation of Philadelphia, PA. Microcrystalline cellulose is typically present in an amount from about 10% to about 20% by weight of the pharmaceutical composition, more typically from about 15% to about 20%, and most typically from about 17% to about 19% microcrystalline cellulose. Microcrystalline cellulose is a fibrous thickening agent typically made by acid hydrolysis of cellulose. The dry ingredients of the decongestant granulation are typically mixed. Usually, the dry ingredients are added to a high shear granulator and mixed for from about 4 minutes to about 6 minutes, such as about 5 minutes. A binding solution is then typically prepared by mixing water and at least one water soluble binder, such as a povidone, including POVIDONE® K-90, which is a polyvinylpyrolidone with a molecular weight of about 90,000. Polyvinylpyrolidone is an essentially linear, non-crosslinked polymer. Usually, polyvinylpyrolidone is the only binder mixed with water to form the binder solution, but mixtures of binders may also conceivably be used in the binder solution. When povidone is used, it is typically included in an amount from about 0.1% to about 4% by weight of the pharmaceutical composition, more typically from about 0.2% to about 0.8%, and most typically from about 0.4% to about 0.6%.

The decongestant granules are then formed by spraying the binder solution onto the mixture of dry ingredients over a period of from about four to about six minutes, typically over an about five minute period. Thereafter, the sprayed dry ingredients are granulated for at least about 15 minutes. The granulation is then typically wet milled using a QUADRO® COMIL® and dried in a fluid bed dryer, typically until LOD % is less than about 3.0%. The granules thereby formed are typically then tested to ensure they pass through a #20 US mesh screen. The granules that will not pass through a #20 US mesh screen are typically milled.

The decongestant granulation is then combined with at least one flow agent, at least one binder, at least one lubricant, and a controlled-release agent, which consists essentially of an ungranulated xanthan gum. Xanthan gum is a natural linear polysaccharide produced by viscous fermentation of the bacterium Xanthomonas campestris. The backbone of the xanthan gum molecule is similar to that of cellulose with side chains attached to alternate glucose residues. The side chains consist of mannose-acetate, mannose, and glucuronic acid. Pyruvate compounds are attached to some single unit side chains by ketal linkages. The molecular weight of xanthan gum is from approximately 2 to about 50 million daltons. Typically, the controlled-release agent of the present invention consists essentially of about 40% to about 60% ungranulated xanthan gum by weight of the pharmaceutical composition, more typically about 40% to about 50%, and most typically about 45% to about 50% ungranulated xanthan gum.

Even at low concentrations, xanthan gum solutions show a high degree of viscosity in comparison with other polysaccharide solutions. Preferably, the ungranulated xanthan gum of the present invention has a viscosity of about 1200 centipoise to about 1600 centipoise.

Xanthan gum is completely soluble in water. However, the time required for full dissolution (the polymer's hydration rate) can be influenced by a number of factors. The ungranulated xanthan gum of the present invention typically has a particle size wherein at least 95% of the particles are about 180 microns or larger.

Applicants have surprisingly discovered that using ungranulated xanthan gum as the sole controlled-release agent results in a 24-hour pseudoephedrine dissolution rate profile. It is presently. believed that the granulated pseudoephedrine and large amounts of ungranulated xanthan gum synergistically work to reduce the pseudoephedrine release rate. The extended release profile is achieved because there is less surface area in contact with stomach and intestinal fluids, thereby slowing down dissolution of the compressed core.

The extended-release core also typically includes at least one core binder beyond those binders already included in the decongestant granules. As with the other binders, the core binder(s) may be any pharmaceutically acceptable binder including microcrystalline cellulose, copolyvidonum, ethyl cellulose, methyl cellulose, stearic acid, povidone; and mixtures thereof; however, copolyvidonum is typically used as the core binder of the present invention. Copolyvidonum is typically present in an amount from about 1% to about 10% by weight of the pharmaceutical composition, more typically from about 1% to about 5% by weight of the pharmaceutical composition, and most typically from about 1% to about 3% copolyvidonum by weight of the pharmaceutical composition

Any pharmaceutically acceptable flow agent such as silicon dioxide, calcium silicate, magnesium silicate, starch, talc, and mixtures thereof may be used as the flow agent of the core. The preferred flow agent of the present invention is a fumed colloidal silicon dioxide such as CAB-O-SIL® M5. Typically, the flow agent is present in the pharmaceutical composition in an amount from about 0.1% to about 1% by weight of the pharmaceutical composition, more typically from about 0.5% to about 1.0% by weight of the pharmaceutical composition, and most typically from about 0.8% to about 0.95% by weight of the pharmaceutical composition.

The extended-release core also typically includes a lubricant. Any pharmaceutically acceptable lubricant may be used in the pharmaceutical composition of the present invention, such as magnesium stearate, calcium stearate, zinc stearate, talc, magnesium lauryl sulfate, sodium benzoate, sodium lauryl sulfate, and glyceryl monostearate. The preferred lubricant is magnesium stearate which is typically present in an amount from about 0.1% to about 1% by weight of the pharmaceutical composition, more typically from about 0.3% to about 0.8% by weight of the pharmaceutical composition, and most typically from about 0.4% to about 0.6% by weight of the pharmaceutical composition. The lubricant is typically a lubricant which will pass through a #30 US mesh screen.

All of the ingredients of the core, except a lubricant (when utilized) are then typically mixed for at least about 10 minutes. A lubricant is then typically added and mixed for an additional at least about 3 minutes. Thereafter, the core is formed by compressing the core ingredients into the desired tablet shape. When the decongestant is a pseudoephedrine salt, the tablet cores typically have a weight of about 950 mg, a thickness of from about 0.270″ to about 0.290″, and a hardness of about 26 Strong-Cobb units (SCU).

The extended-release core is then typically coated with an immediate-release coating composition that generally includes an antihistamine, typically a non-sedative antihistamine, such as loratadine or desloratadine, at least one coating agent, such as OPADRY® II White, and a surfactant. Loratadine is a tricyclic antihistamine, which has a selective and peripheral H1-antagonist action. It has a long-lasting effect and does not cause drowsiness because it does not readily enter the central nervous system. Loratadine is rapidly absorbed from the gastrointestinal tract and has rapid first-pass hepatic metabolism. Loratadine is almost totally bound to plasma proteins. Its metabolite, desloratadine, is also active, but binds to plasma proteins only moderately. The half-life of loratadine is typically about 8 hours, and the half-life of metabolite is typically about 28 hours. Typically, the non-sedating antihistamine, such as loratadine, is present in the immediate-release coating of the present invention in an amount from about 0.1% to about 1% by weight of the pharmaceutical composition, more typically from about 0.5% to about 1.0% by weight of the pharmaceutical composition, and most typically from about 0.9% to about 1% by weight of the pharmaceutical composition.

The immediate-release coating composition also typically includes at least one coloring or coating agent, such as OPADRY® II White, which contains talc, titanium dioxide, polyvinyl alcohol, and polyethylene glycol. Other suitable coating agents include polyvinyl alcohol, titanium dioxide, polyethylene glycol, sodium lauryl sulfate, cellulose acetate, cellulose acetate phthalate, cetyl alcohol, ethyl cellulose, glycerin, hydroxyethyl cellulose, hydroxyethylmethyl cellulose, methyl cellulose, tributyl citrate, triethyl citrate and talc. The immediate-release coating composition may also include a buffering agent such as magnesium hydroxide (Mg(OH)2), and sodium hydroxide (NaOH). The immediate-release coating composition of the present invention may also contain a surfactant such as sodium lauryl sulfate. The immediate-release coating composition typically includes the coating or coloring agent(s) and any surfactant utilized in an amount from about 2% to about 20% by weight of the pharmaceutical composition, more typically from about 3% to about 10% by weight of the pharmaceutical composition, and most typically from about 4.0% to about 6.0% by weight of the pharmaceutical composition. The immediate-release coating composition is typically spray coated onto the compressed core.

Once the immediate-release coating composition has been applied to the compressed core to form a composite core, an optional finish coat may be applied to the outer surface of the newly formed composite core. While not required, the finish coating is usually applied. When the finish coating is utilized, it is typically applied by spraying the finish coating onto the outer surface of the composite core. The finish coating typically includes a solution of water and a coloring or coating agent such as an OPADRY®, in particular OPADRY® II White, sold by Coloron Corp. Both the immediate-release coating and finish coating are typically applied using an ACCELA-COTA® machine.

Pseudoephedrine sulfate and loratadine tablets produced according to the above yielded the in vitro pseudoephedrine release rates given in Table 1 under the conditions set out below.

TABLE 1
(Percent Release of Pseudoephedrine)

| Tablet # | 1 hr | 2 hr | 4 hr | 6 hr | 8 hr | 10 hr | 12 hr | 16 hr | 20 hr | 24 hr |
|---|---|---|---|---|---|---|---|---|---|---|
| 1 | 16 | 25 | 39 | 50 | 60 | 68 | 76 | 87 | 92 | 99 |
| 2 | 15 | 23 | 37 | 47 | 57 | 63 | 70 | 80 | 87 | 91 |
| 3 | 15 | 24 | 37 | 47 | 57 | 64 | 70 | 80 | 87 | 92 |
| 4 | 16 | 25 | 39 | 51 | 61 | 69 | 76 | 86 | 94 | 99 |
| 5 | 16 | 26 | 40 | 51 | 59 | 67 | 73 | 82 | 91 | 95 |
| 6 | 15 | 24 | 38 | 49 | 58 | 65 | 73 | 82 | 88 | 95 |
| Mean | 16 | 24 | 38 | 49 | 58 | 66 | 73 | 83 | 90 | 95 |
| Low | 15 | 23 | 37 | 47 | 57 | 63 | 70 | 80 | 87 | 91 |
| High | 16 | 26 | 40 | 51 | 61 | 69 | 76 | 87 | 94 | 99 |
| Percent Relative | 3.5 | 4.3 | 3.2 | 3.7 | 2.8 | 3.6 | 3.7 | 3.6 | 3.2 | 3.5 |
| Standard | | | | | | | | | | |
| Deviation | | | | | | | | | | |

Media: 900 ml, pH 7.5, 0.1 M phosphate buffer
Apparatus: USP II at 50 RPM
Temperature: 37° C.±5° C.

The invention claimed is:

1. A pharmaceutical composition comprising:
a controlled release core comprising: an ungranulated xanthan gum in an amount of from about 40% to about 60% by weight of the composition; a decongestant chosen from the group consisting of pseudoephedrine salt and a phenylephrine salt wherein the decongestant is present in an amount of from about 20% to about 30% by weight of the composition; microcrystalline cellulose in an amount of from about 10% to about 20% by weight of the composition; copolyvidonum in an amount of from about 1% to about 10% by weight of the composition; povidone in an amount of less than about 1% by weight of the composition; amorphous silicon dioxide in an amount of less than about 1% by weight of the composition; and magnesium stearate in an amount of less than about 1% by weight of the composition; and
an immediate release coating composition disposed on the core comprising polyvinyl alcohol in an amount of from about 1% to about 10% by weight of the composition; titanium dioxide in an amount of about 1% to about 10% by weight of the composition; polyethylene glycol in an amount of less than about 1% by weight of the composition; sodium lauryl sulfate in an amount of less than about 1% by weight of the composition; talc in an amount of less than about 1% by weight of the composition; loratadine in an amount of less than about 1% by weight of the composition.

2. The pharmaceutical composition of claim 1, wherein the controlled release core comprises:
an ungranulated xanthan gum in an amount of from about 45% to about 50% by weight of the composition;
pseudoephedrine sulfate in an amount of from about 22% to about 24% by weight of the composition;
microcrystalline cellulose in an amount of from about 17% to about 19% by weight of the composition;
copolyvidonum in an amount of from about 1% to about 3% by weight of the composition.

3. The pharmaceutical composition of claim 2, wherein the ungranulated xanthan gum has a particle size wherein at least 95% of the particles are about 180% or larger.

4. The pharmaceutical composition of claim 3, wherein the ungranulated xanthan gum is the sole controlled-release agent.

5. The pharmaceutical composition of claim 1, wherein the ungranulated xanthan gum is the sole controlled-release agent.

6. The pharmaceutical composition of claim 1, wherein the decongestant comprises a decongestant granulation.

7. A pharmaceutical composition comprising: a pharmaceutically effective amount of a pharmaceutically effective amount of a decongestant, one or more binders, and a controlled release agent consisting essentially of ungranulated xanthan gum.

8. The pharmaceutical composition of claim 7, wherein the controlled release agent consists of ungranulated xanthan gum and provides controlled release of the decongestant and wherein the decongestant comprises a decongestant chosen from the group consisting of a pseudoephedrine salt and a phenylephrine salt.

9. The pharmaceutical composition of claim 7, further comprising a non-sedative antihistamine, wherein the non-sedative antihistamine comprises a non-sedative antihistamine chosen from the group consisting of an ethylenediamine; an ethanolamine; an alkylamine; a piperazine; a tricyclic antihistamine compound; acrivastine; astemizole; cetirizine; levocetirizine; fexofenadine; loratadine; desloratadine; mizolastine; and terfenadine.

10. The pharmaceutical composition of claim 9, wherein the composition comprises:
an extended release core and an immediate release coating wherein the core comprises:
a decongestant granulation comprising the decongestant and a binder; and
the controlled release agent, and
wherein the immediate release coating is disposed on the core and comprises the non-sedating antihistamine and at least one coating agent.

11. The pharmaceutical composition of claim 10, further comprising a finish coating disposed on the immediate release coating and wherein the finish coating comprises water and agent selected from the group consisting of a coloring agent and a coating agent and wherein the ungranulated xanthan gum has a particle size wherein at least 95% of the particles are about 180% or larger.

12. The pharmaceutical composition of claim 10, wherein the controlled release agent consisting essentially of ungranulated xanthan gum is present in an amount of from about 40% to about 60% by weight of the composition; the decongestant is present in an amount of from about 20% to about 30% by weight of the composition; the one or more binders are present in an amount of from about 10% to about 20% by weight of the composition; the non-sedative antihistamine is present in an amount of less than about 1% by weight of the composition.

13. The pharmaceutical composition of claim 12, wherein the immediate release coating further comprises an agent comprising talc, titanium dioxide, polyvinyl alcohol, and polyethylene glycol and optionally a surfactant wherein the agent and the surfactant comprise from about 2% to about 20% by weight of the composition.

14. The pharmaceutical composition of claim 13, wherein the core further comprises a flow agent in an amount of less than about 1% of the composition and a lubricant in an amount of less than.about 1% of the composition.

15. The pharmaceutical composition of claim 7, wherein the decongestant comprises pseudoephedrine sulfate and the composition has an in vitro release profile wherein from 15% to 16% of the decongestant is released after one hour, from 23% to 26% is released after two hours, from 37% to 40% is released after four hours, from 47% to 51% is released after six hours, from 57% to 61% is released after eight hours, from 63% to 69% is released after ten hours, from 70% to 76% is released after 12 hours, from 80% to 87% is released after 16 hours, from 87% to 94% is released after 20 hours, and from 91% to 99% is released after 24 hours.

16. A pharmaceutical composition comprising:
a controlled release core comprising about 40% to about 60% by weight of the composition wherein ungranulated xanthan gum is the sole controlled release agent; a decongestant in an amount of from about 20% to about 30% by weight of the composition; at least one binder in an amount of from about 10% to about 30% by weight of the composition; a flow agent in an amount of less than about 1% by weight of the composition; and a lubricant in an amount of less than about 1% by weight of the composition; and
a coating composition comprising a coating agent; and about less than 1% by weight of a non-sedative antihistamine in an amount of less than about 1% by weight of the composition.

17. The pharmaceutical composition of claim 16, wherein the ungranulated xanthan gum provides controlled release of the decongestant and the decongestant comprises a decongestant chosen from the group consisting of a pseudoephedrine salt and a phenylephrine salt.

18. The pharmaceutical composition of claim 17, wherein the non-sedative antihistamine comprises a non-sedative antihistamine chosen from the group consisting mepyramine, pyrilamine, antazoline, diphenhydramine, carbinoxamine, doxylamine, clemastine, dimenhydrinate, pheniramine, chlorphenamine, chlorpheniramine, dexchlorphenamine, brompheniramine, triprolidine, hydroxyzine, meclizine, promethazine, alimemazine, trimeprazine, cyproheptadine, azatadine, acrivastine, astemizole, cetirizine, levocetirizine, fexofenadine, loratadine, desloratadine, mizolastine, and terfenadine.

19. The pharmaceutical composition of claim 18, wherein the non-sedative antihistamine comprises a non-sedative antihistamine chosen from the group consisting of desloratadine and loratadine.

20. A process for making an extended release decongestant/non-sedating antihistamine tablet comprising the steps of:
mixing a decongestant chosen from the group consisting of a pseudoephedrine salt and a phenylephrine salt and present in an amount of from about 20% to about 30% by weight of the tablet and microcrystalline cellulose in an amount of from about 10% to about 20% by weight of the tablet to form a decongestant/microcrystalline cellulose mixture;
spraying the decongestant/microcrystalline cellulose mixture with a povidone in an amount of from about less than 1% by weight of the tablet to form a decongestant/microcrystalline cellulose/povidone mixture;
granulating the decongestant/microcrystalline cellulose/povidone mixture to form granulated decongestant particles;
combining the granulated decongestant particles with ungranulated xanthan gum in an amount of from about 40% to about 60% by weight of the tablet; copolyvidonum in an amount of from about 1% to about 10% by weight of the tablet; an amorphous silicon dioxide in an amount of less than about 1% by weight of the tablet; and magnesium stearate in an amount of less than about 1% by weight of the tablet to form a core mixture;
compressing the core mixture to form a compressed, extended release core;
applying a coating to the compressed, extended release core to form the extended release decongestant/non-sedating antihistamine tablet wherein the coating composition comprises: polyvinyl alcohol in an amount of from about 1% to about 10% by weight of the tablet; a titanium dioxide in an amount of from about 1% to about 10% by weight of the tablet; polyethylene glycol in an amount of less than about 1% by weight of the tablet; sodiuin lauryl sulfate in an amount of less than about 1% by weight of the tablet; talc in an amount of less than about 1% by weight of the tablet; and a non-sedating antihistamine comprising a non-sedating antihistamine chosen from the group consisting of desloratadine and loratadine wherein the non-sedating antihistamine is present in an amount of less than about 1% by weight of the tablet.

21. A process for making an extended release tablet comprising the steps of:
granulating a decongestant and a binder to form a granulated decongestant;
combining the granulated decongestant with ungranulated xanthan gum, at least one additional binder, a flow agent, and a lubricant to form a core mixture;
compressing the core mixture to form an extended release core;
coating the extended release core with a coating composition comprising a non-sedative antihistamine.

22. The process of claim 21 wherein the decongestant comprises a phenylephrine salt, the non-sedative antihistamine comprises a non-sedative antihistamine chosen from the group consisting of desloratadine and loratadine, the coating composition further comprises a coating agent, and the ungranulated xanthan gum is the sole controlled release agent.
